(19) United States
(12) Patent Application Publication
(10) Pub. No.: US 2003/0017594 A1
(43) Pub. Date: Jan. 23, 2003

(54) RecT or RecET cloning and subcloning method

(76) Inventors: Zhang Youming, Dresden (DE); Stewart A. Francis, Waldstadt (DE); Muijrers P. P. Joep, Amsterdam (NL)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/199,478

(22) Filed: Jul. 22, 2002

(30) Foreign Application Priority Data
Jul. 20, 2001 (EP) 01 117 529.6

(51) Int. Cl. C12N 15/85; C12N 5/06

(52) U.S. Cl. 435/455; 435/320.1; 435/325

(57) ABSTRACT

The invention refers to an improved method for DNA cloning and subcloning using Rec T or RecET-mediated homologous recombination. Further, novel reagent kits suitable for carrying out the method are provided.

The invention refers to an improved method for DNA cloning and subcloning using RecT or RecET-mediated homologous recombination. Further, novel reagent kits suitable for carrying out the method are provided.

WO99/29837 discloses a method for cloning DNA molecules in cells via a RecET-dependent mechanism of homologous recombination. Modifications of this method are described in WO00/26396 and WO01/04288. GB application 0 103 276.2 discloses a method for cloning DNA molecules in cells via a RecT-dependent mechanism of homologous recombination. These documents are herein incorporated by reference.

Although the RecET cloning method represents a considerable progress in the field of recombinant DNA technology and has meanwhile been applied on a very large scale, in some cases there are still problems with regard to the recombination efficiency. Particularly, when employing chemically competent host cells often a relatively small number of recombinant clones (or none at all) is obtained.

Muyrers et al. (Genes and Development 14 (2000), 1971-1982) describe that the RecET-mediated recombination is RecA-independent and that the recombination efficiency is not increased in electrocompetent RecA-positive host cells compared to electrocompetent RecA-negative host cells.

Surprisingly, it was found in subsequent studies that the efficiency of RecT- and RecET-mediated homologous recombination in chemically competent host cells is improved to a large extent when RecA-positive host cells are used.

Due to this high efficiency, chemically competent cells can be used instead of electrocompetent cells. As chemically competent cells are much easier to prepare than electrocompetent cells, this is a very significant improvement.

Furthermore, it was found that an overexpression of RecA particularly under control of a regulatable promoter increases the efficiency of RecT and RecET cloning also for electrocompetent host cells.

Thus, a simplified RecT- or RecET-cloning or subcloning method and reagent kits suitable for carrying out this method are provided.

A first subject matter of the present invention is a method for cloning DNA molecules in cells comprising the steps of a) providing means for performing homologous recombination via a RecT dependent mechanism within a host cell and/or in vitro, b) i) contacting in said host cell a first DNA molecule which is capable of being replicated in said host cell with a second DNA molecule under conditions which favour homologous recombination between said first and second DNA molecules and/or ii) contacting in vitro a first DNA molecule which is capable of being replicated in said host cell with a second DNA molecule under conditions which favour homologous recombination between said first and second DNA molecules and introducing recombined DNA molecules into said host cell and c) selecting a host cell in which homologous recombination between said first and second DNA molecules has occurred, wherein a chemically competent host cell and a RecA activity are provided.

Preferably means are provided of performing homologous recombination via a RecET-dependent mechanism within a host cell and/or in vitro.

The chemically competent host cell suitable for the method of the present invention preferably is a bacterial cell, e.g. a gram-negative or gram-positive bacterial cell. More preferably, the host cell is an enterobacterial cell, such as Salmonella, Klebsiella or Escherichia, a gram-positive Bacillus cell or a gram-negative Acenitobacter cell. Most preferably, the host cell is an Escherichia coli cell. The prokaryotic host cell can be made competent, e.g. capable of internalizing DNA-molecules, according to known methods, such as treatment with calcium chloride, rubidium chloride, glycerol, dimethyl sulfoxide or any combination thereof. More preferably, the cell has been made competent by treatment with calcium chloride or rubidium choride.

On the other hand, the host cell may also be a eukaryotic cell, such as a yeast cell, an insect cell, or a mammalian cell. Chemically competent eukaryotic cells are obtainable by any suitable method, e.g. by a treatment with calcium phosphate, DEAE-dextran, lipids or any combination thereof. More preferably, the cell has been made competent by treatment with calcium phosphate.

According to the present invention, the recE and recT genes are preferably selected from E.coli recE and rect genes or from bacteriophage genes, particularly the lambda red α and red β genes or from recombination-competent fragments thereof. The nucleotide sequences of these genes, and functionally active variants thereof, e.g. deletion or hybridization variants and the amino acid sequences encoded thereby are disclosed in WO99/29837, and are thus part of the present application. Furthermore, the invention comprises the use of functionally related genes, e.g. from lambdoid phages, such as phage P22.

In a preferred embodiment of the invention a host cell is used which is capable of expressing a recT gene and optionally a recE gene, i.e. the host cell contains a nucleic acid sequence encoding the recE gene and optionally the recT gene.

The host cell may comprise the recE and recT genes located on the host cell chromosome or on non-chromosomal DNA, preferably on a vector, e.g. a plasmid or a chromosomal vector. In a preferred case the recE and/or recT gene products are expressed from regulatable promoters, such as the arabinose-inducible BAD promoter or the lac promoter, or from nonregulatable promoters. Alternatively, the recE and recT genes are expressed on a polycistronic mRNA from a single regulatable or nonregulatable promoter.

Alternatively or additionally, the RecT protein and/or the RecE protein may be contacted outside the host cell with the first and/or the second DNA molecule and then introduced into the host cell. For example, the RecT protein and optionally the RecE protein and/or optionally the RecA protein and/or optionally the RecBCD protein may be preincubated with the first and/or the second DNA molecule before introduction into the host cell. In a specific embodiment a joint molecule is introduced into the host cell which is a product of an in vitro preincubation of a protein, e.g. a RecT and optionally a RecE plus the first and the second DNA molecule. In a further preferred embodiment a coated molecule is introduced into the host cell which is the product of an in vitro incubation with a protein, e.g. RecT and optionally RecE and/or optionally RecA and/or optionally RecBCD plus the second DNA molecule. It should be noted that the in vitro preincubation may be combined with the use of a cell capable of expressing recE and/or recT genes and optionally recA genes and/or optionally recBCD genes in order to enhance the cloning efficiency. The use of joint and coated molecules is explicitly disclosed in GB application 0103276.2.

The present invention requires a RecA activity, e.g. by using a host cell capable of expressing the recA gene. More preferably, the recA gene is a bacterial gene, such as E.coli recA gene (Kowalczykowski et al., Microbio Rev. (1994) 58(3):401-65). However, other bacterial recA genes are also suitable, such as recA homologues present in Salmonella, Klebsiella or Escherichia, Bacillus or Acenitobacter. Also, bacteriophage-derived homologues, such as UvsX (Bianco et al., 1998) are suitable. Alternatively, the eukaryotic recA homolog RAD51 or homologues thereof, such as DMC1 (Grushcow et al., Genetics (1999) 153(2):607-20), RAD51 (Bianco et al., Front Biosci. (1998) 3:D570-603), RAD51B, RAD51C, RAD51D, XRCC2, XRCC3 (Grushcow et al., Genetics (1999) 153(2):607-20; Takata et al., Mol Cell Biol (2001) 21(8):2858-66) may be used. The host cell may comprise the recA gene located on the chromosome. Preferably, however, the host cell is transformed with a vector, e.g. a plasmid capable of expressing the recA gene. The recA gene may be under control of a regulatable or a non-regulatable promoter. More preferably, the recA gene is located on a vector together with the recE and the recT gene. In that case, the recA gene may be co-expressed with the recT gene and optionally the recE gene, e.g. under control of a single regulatable or nonregulatable promoter. Most preferably, the host cell is capable of overexpressing the recA gene.

Alternatively or additionally, the RecA activity may be provided by in vitro preincubating a RecA protein with the first and/or second DNA molecule and then introducing the resulting product into the host cell, e.g. as a coated and/or joint molecule as described above.

In a preferred embodiment of the present invention a RecBC inhibitor activity is provided, e.g. by using a host cell capable of expressing a recBC inhibitor gene. A suitable example of such a recBC inhibitor is the lambda red γ gene which is disclosed in WO99/29837, or a functional equivalent thereof which for example may be obtained from other bacteriophages, particularly lambdoid bacteriophages, such as from phage P22 (Murphy, J. Biol. Chem. 269 (1994) 22507-22516).

The host cell may comprise the recBC inhibitor gene located on the host cell chromosome or on a non-chromosomal DNA, preferably on a vector, e.g. a plasmid. In a preferred case, the recBC inhibitor is expressed from a regulatable promoter. The co-expression of a recBC inhibitor gene in the host cell leads to a significant improvement of cloning efficiency as described in WO99/29837. In a particularly preferred embodiment the recBC inhibitor gene is co-expressed with the recT, recA and optionally recE genes, e.g. under control of a single regulatable promoter.

Alternatively or additionally, the RecBC inhibitor can be preincubated with the first and/or second DNA molecule which is then introduced into the host cell, e.g. as a coated and/or joint molecule as described above.

The cloning method according to the present invention employs a homologous recombination between a first DNA molecule and a second DNA molecule in vitro and/or in vivo. The first DNA molecule is preferably a double-stranded DNA molecule that carries an origin of replication which is operative in the host cell, e.g. an E.coli replication origin. The first DNA molecule can be any extrachromosomal DNA molecule containing an origin of replication which is operative in said host cell, e.g. a plasmid including single, low, medium or high copy plasmids or other extrachromosomal circular DNA molecules based on cosmid, P1, BAC or PAC vector technology. Examples of such vectors are described, for example, by Sambrook et al. (Molecular Cloning, Laboratory Manual, 2nd Edition (1989), Cold Spring Harbor Laboratory Press) and loannou et al. (Nature Genet. 6 (1994), 84-89) or references cited therein. The first DNA molecule is preferably circular. The first DNA molecule can also be a host cell chromosome, particularly the E.coli chromosome. Preferably, the first DNA molecule is a double-stranded DNA molecule.

The second DNA molecule is preferably a linear DNA molecule and comprises at least two regions of sequence homology, preferably of sequence identity for allowing homologous recombination with the first DNA molecule. These homology or identity regions are preferably at least 15 nucleotides each, more preferably at least 20 nucleotides and, most preferably, at least 30 nucleotides each. Especially good results were obtained when using sequence homology regions having a length of about 40 or more nucleotides, e.g. 60 or more nucleotides. The two sequence homology regions can be located on the linear DNA fragment so that one is at one end and the other is at the other end, however they may also be located internally. Preferably, also the second DNA molecule is a double-stranded DNA molecule. It should be noted, however, that also single-stranded DNA molecules can be used. Furthermore, DNA molecules which have been in vitro preincubated with proteins such as RecE and/or RecT proteins and/or RecA proteins and/or RecBCD inhibitors are suitable for carrying out the invention.

Thus the invention also comprises a combination of in vitro incubation with RecE and/or RecT and/or RecA and/or RecBCD inhibitor, followed by introducing such in vitro incubated molecules into a bacterial strain that expressed none, one, two, three or four of these classes of proteins.

The two sequence homology regions are chosen according to the experimental design. There are no limitations on which regions of the first DNA molecule can be chosen for the two sequence homology regions located on the second DNA molecule, except that the homologous recombination event may not disrupt the origin of replication of the first DNA molecule, unless the second DNA molecule contains an origin of replication. The sequence homology regions can be interrupted by non-identical sequence regions as long as sufficient sequence homology is retained for the homologous recombination reaction. By using sequence homology arms having non-identical sequence regions compared to the target site mutations such as substitutions, e.g. point mutations, insertions and/or deletions may be introduced into the target site by ET cloning.

The second foreign DNA molecule which is to be cloned in the bacterial cell may be derived from any source. For example, the second DNA molecule may be a synthetic oligonucleotide, or may be synthesized by a nucleic acid amplification reaction such as a PCR where both of the DNA oligonucleotides used to prime the amplification contain in addition to sequences at the 3′-ends that serve as a primer for the amplification, one or the other of the two homology regions. Using oligonucleotides of this design, the DNA product of the amplification can be any DNA sequence suitable for amplification and will additionally have a sequence homology region at each end.

A specific example of the generation of the second DNA molecule is the amplification of a gene that serves to convey a phenotypic difference to the bacterial host cells, in particular, antibiotic resistance. A simple variation of this procedure involves the use of oligonucleotides that include other sequences in addition to the PCR primer sequence and the sequence homology region. A further simple variation is the use of more than two amplification primers to generate the amplification product. A further simple variation is the use of more than one amplification reaction to generate the amplification product. A further variation is the use of DNA fragments obtained by methods other than PCR, for example, by endonuclease or restriction enzyme cleavage to linearize fragments from any source of DNA. Furthermore, chemically synthesized nucleic acid molecules, e.g. oligonucleotides may be used. Finally, also coated and/or joint molecules as described above can be used.

It should be noted that the second DNA molecule is not necessarily a single species of DNA molecule. It is of course possible to use a heterogenous population of second DNA molecules, e.g. to generate a DNA library, such as a genomic or cDNA library.

Generally the DNA cloning and subcloning can comprise cloning by mixing a first DNA molecule (e.g. circular or linear, pure or a mixture such as genomic DNA) with a second DNA molecule, a linear vector.

In a further embodiment the recT or recET mediated recombination comprises a sub-cloning step wherein the first DNA molecule is preferably a vector in linearized configuration. This linearized vector contains termini having defined sequences. The second DNA molecule which is preferably a linear DNA molecule comprises two regions of sequence homology, preferably of sequence identity to the termini on the first linearized DNA molecule. In a particular embodiment a pair of adaptor oligonucleotides is used, each comprising a sequence homology region to the first and to the second DNA molecule. In this embodiment the second DNA molecule need not comprise regions of sequence homology to regions on the first DNA molecule, but to the adaptor oligonucleotide which, in turn, has a further region of sequence homology enabling homologous recombination with the first DNA molecule.

The method of the present invention comprises the contacting of the first and second DNA molecules in vivo. In one embodiment of the present invention the second DNA fragment is transformed into a host cell, e.g. a bacterial strain that already harbors the first vector DNA molecule. In a different embodiment, the second DNA molecule and the first DNA molecule are mixed together in vitro before co-transformation into the host cell. In a variation of this embodiment, the first and/or second DNA molecule can be preincubated in vitro with RecT, and/or RecA, and/or RecE, and/or the RecBC inhibitor.

After contacting the first and second DNA molecules under conditions which favour homologous recombination between first and second DNA molecules via the T or ET cloning mechanism a host cell is selected, in which homologous recombination between said first and second DNA molecules has occurred. This selection procedure can be carried out by several different methods including those described in detail in WO99/29837 and WO01/04288.

In a first selection method a second DNA fragment is employed which carries a gene for a marker placed between the two regions of sequence homology wherein homologous recombination is detectable by expression of the marker gene. The marker gene may be a gene for a phenotypic marker, e.g. an antibiotic resistance gene, a deficiency complementing gene or a detectable gene, which is not expressed in the host or from the first DNA molecule. Upon recombination by T or ET cloning, the change in phenotype of the host strain conveyed by the stable acquisition of the second DNA fragment identifies the T or ET cloning product.

In a second selection method the insertion of the second DNA fragment into the first DNA molecule by ET cloning alters the expression of a marker present on the first DNA molecule. In this embodiment the first DNA molecule contains at least one marker gene between the two regions of sequence homology and homologous recombination may be detected by an altered expression, e.g. lack of expression of the marker gene.

In a third selection method the integration of the second DNA fragment into the first DNA molecule by T or ET cloning removes a target site for a site specific recombinase, termed here an RT (for recombinase target) present on the first DNA molecule between the two regions of sequence homology. A homologous recombination event may be detected by removal of the target site.

In the absence of the T or ET cloning product, the RT is available for use by the corresponding site specific recombinase. The difference between the presence or not of this RT is the basis for selection of the T or ET cloning product. In the presence of this RT and the corresponding site specific recombinase, the site specific recombinase mediates recombination at this RT and changes the phenotype of the host so that it is either not able to grow or presents a readily observable phenotype. In the absence of this RT, the corresponding site specific recombinase is not able to mediate recombination.

In another preferred case, the RT to be removed by T or ET cloning of the second DNA fragment is anywhere on a first episomal DNA molecule and the episome carries an origin of replication incompatible with survival of the bacterial host cell if it is integrated into the host genome. In this case the host genome carries a second RT, which may or may not be a mutated RT so that the corresponding site specific recombinase can integrate the episome, via its RT, into the RT sited in the host genome. Other preferred RTs include RTs for site specific recombinases of the resolvase/transposase class. RTs include those described from existing examples of site specific recombination as well as natural or mutated variations thereof.

The preferred site specific recombinases include Cre, FLP, Kw or any site specific recombinase of the integrase class. Other preferred site specific recombinases include site specific recombinases of the resolvase/transposase class.

There are no limitations on the method of expression of the site specific recombinase in the host cell. In a preferred method, the expression of the site specific recombinase is regulated so that expression can be induced and quenched according to the optimisation of the ET cloning efficiency. In this case, the site specific recombinase gene can be either integrated into the host genome or carried on an episome. In another preferred case, the site specific recombinase is expressed from an episome that carries a conditional origin of replication so that it can be eliminated from the host cell.

In another preferred case, at least two of the above three selection methods are combined. A particularly preferred case involves a two-step use of the first selection method above, followed by use of the second selection method. This combined use requires, most simply, that the DNA fragment to be cloned includes a gene, or genes that permits the identification, in the first step, of correct T or ET cloning products by the acquisition of a phenotypic change. In a second step, expression of the gene or genes introduced in the first step is altered so that a second round of T or ET cloning products can be identified. In a preferred example, the gene employed is the tetracycline resistance gene and the first step T or ET cloning products are identified by the acquisition of tetracycline resistance. In the second step, loss of expression of the tetracycline gene is identified by loss of sensitivity to nickel chloride, fusaric acid or any other agent that is toxic to the host cell when the tetracycline gene is expressed. Other preferred examples are counter selectable genes including the use of rpsL and sacB genes, in which loss of expression is identified by loss of sensitivity to streptomycin, sucrose, or any other reagents that is toxic to the host cell when the rpsL or sacB gene is expressed. It should be noted that also other counter selectable genes can be used. This two-step procedure permits the identification of T or ET cloning products by first the integration of a gene that conveys a phenotypic change on the host, and second by the loss of a related phenotypic change, most simply by removal of some of the DNA sequences integrated in the first step. Thereby the genes used to identify T or ET cloning products can be inserted and then removed to leave T or ET cloning products that are free of these genes.

It should be noted, however, that selection step (c) may comprise a screening method without employing any selection marker at all. This screening method may comprise assaying single clones, e.g. via a nucleic acid amplification such as PCR, or via sequencing or colony hybridization or any combination of these methods in order to identify clones having the desired sequence. The high efficiency of the method of the present invention allows the identification of host cells in which homologous recombination has occured without undue effort.

A further subject matter of the invention is the use of cells, preferably bacterial cells, most preferably E.coli cells capable of expressing the recT gene, the recA gene and optionally the recE gene as a chemically competent host cell for a cloning method involving homologous recombination.

Still a further subject matter of the invention is a method for cloning DNA molecules in cells comprising the steps of:

a) providing means for performing homologous recombination via a RecT dependent mechanism within a host cell and/or in vitro,

b) i) contacting in said host cell a first DNA molecule which is capable of being replicated in said host cell with a second DNA molecule under conditions which favour homologous recombination between said first and second DNA molecules and/or

ii) contacting in vitro a first DNA molecule which is capable of being replicated in said host cell with a second DNA molecule under conditions which favour homologous recombination between said first and second DNA molecules and introducing recombined DNA molecules into said host cell, and

c) selecting a host cell in which homologous recombination between said first and second DNA molecules has occurred,

wherein a RecA activity is provided.

In this embodiment the host cell may be a chemically competent or a electrocompetent host cell. The RecT protein, the RecA protein and optionally the RecE protein are provided within the host cell and/or in vitro. Preferably, the host cell is capable of expressing the recT gene, the recA gene and optionally the recE gene. More preferably, the host cell is capable of overexpressing the recA gene, i.e. expressing the recA gene in an amount which is higher than the expression of the endogeneous chromosomal recA locus. It should be noted that the RecT protein, the RecA protein and/or the RecE protein may be contacted outside the host cell, e.g. by a preincubation step, with the first and/or second DNA molecule and then introduced into the host cell, e.g. as coated and/or joint molecule as described above.

The recE, recT and recA genes are preferably under control of a regulatable promoter. More preferably, the recE, recT and recA genes are located on at least one extra-chromosomal vector. This vector may have a temperature-sensitive origin. Further, we refer to the above disclosure of other embodiments of the present invention.

Still a further subject matter of the invention is a vector system capable of expressing the recT gene, the recA gene and optionally the recE gene in a host cell and its use for a cloning method involving homologous recombination. Preferably, the vector system is also capable of expressing a recBC inhibitor gene as defined above, e.g. the λ red γ gene. The vector system may comprise one or several vectors. The recT gene and the recA gene and optionally the recE gene and/or recBC inhibitor gene are preferably located on a single vector and more preferably under control of a regulatable promoter which may be the same for both genes or a single promoter for each gene. Especially preferred is a vector system which is capable of overexpressing the recT gene versus the recE gene.

A still further subject matter of the invention is a reagent kit for recET cloning comprising

(a) a host cell, preferably a chemically competent host cell,

(b) means for providing RecT and RecA activity and optionally RecE activity (i) within said host cell, e.g. by expressing the recT gene, the recA gene and optionally the recE gene in said host cell, e.g. by a vector system and/or by (ii) in vitro, e.g. preincubating the proteins outside said host cell with DNA molecules, and

(c) a recipient cloning vehicle, e.g. a vector, capable of being replicated in said cell.

On the one hand, the recipient cloning vehicle which corresponds to the first DNA molecule of the process of the invention can already be present in the bacterial cell. On the other hand, it can be present separated from the bacterial cell.

The reagent kit furthermore contains, preferably, means for providing RecBC inhibitor and/or a site specific recombinase activity within said host cell, e.g. by expressing a recBC inhibitor gene and/or a site specific recombinase activity within said host cell and/or in vitro, e.g. by preincubating the proteins outside said host cell with DNA molecules, in particular, when the recipient T or ET cloning product contains at least one site specific recombinase target site. Moreover, the reagent kit can also contain DNA molecules suitable for use as a source of linear DNA fragments used for T or ET cloning, preferably by serving as templates for PCR generation of the linear fragment, also as specifically designed DNA vectors from which the linear DNA fragment is released by restriction enzyme cleavage, or as prepared linear fragments included in the kit for use as positive controls or other tasks. Moreover, the reagent kit can also contain nucleic acid amplification primers comprising a region of homology to said vector. Preferably, this region of homology is located at the 5′-end of the nucleic acid amplification primer. Finally, the kit can contain at least one of RecT, RecA, RecE, RecBC inhibitors and site specific recombinase as protein extract or as at least partially purified protein.

The invention is further illustrated by the following Figures.

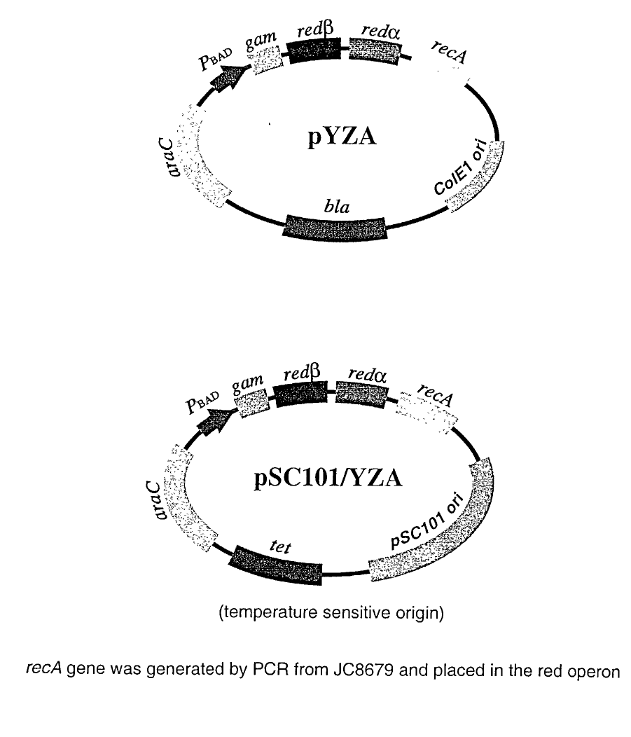

FIG. 1

Two preferred embodiments of vectors for RecETA-cloning are shown by diagram. The vector pYZA comprises the redα, redβ and redγ gene, as well as the recA gene under control of the regulatable BAD promoter. Further, the vector comprises the araC gene and the β lactamase (bla) resistance gene and the ColE1 origin of replication.

The vector pSC101/YZA comprises the tetracyclin resistance gene and the temperature sensitive pSC101 origin of replication.

The recA gene was generated by PCR from E.coli strain JC879 (Gillen et al., J. Bacteriol. 145 (1981), 521-532) and inserted into the red operon.

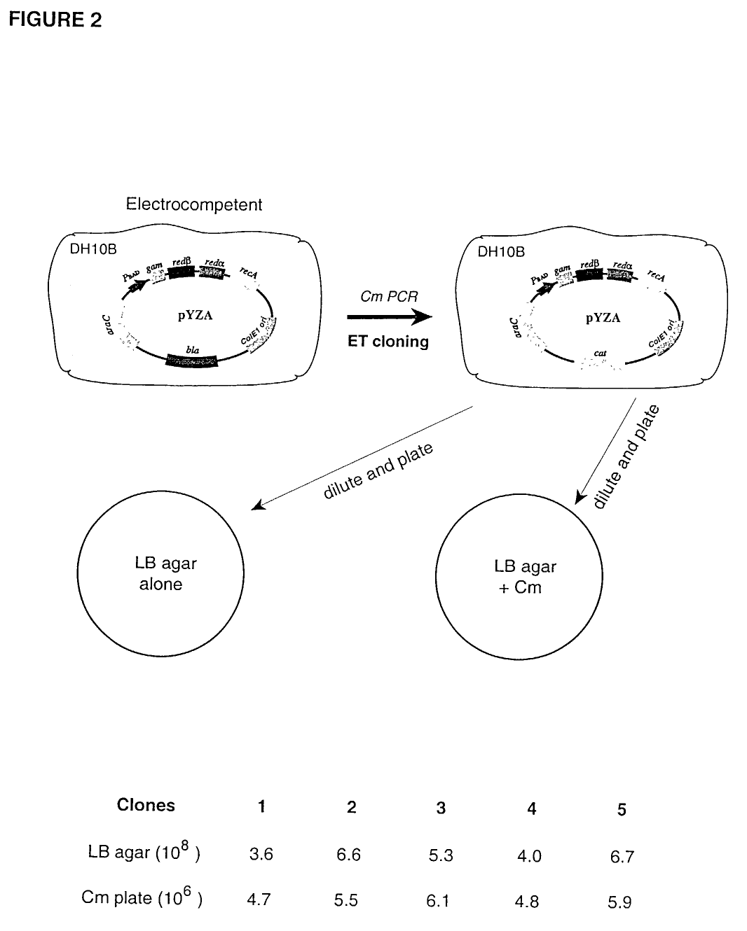

FIG. 2

Electrocompetent E.coli DH10B (Lorow, D., and Jesse, J. Focus (1990) 12:19; Research Genetics) cells were prepared. A PCR product of the chloramphenicol (Cm) resistance gene flanked by homology arms (50 nt) to replace the ampicillin resistance gene was introduced into the competent cells. The results are shown.

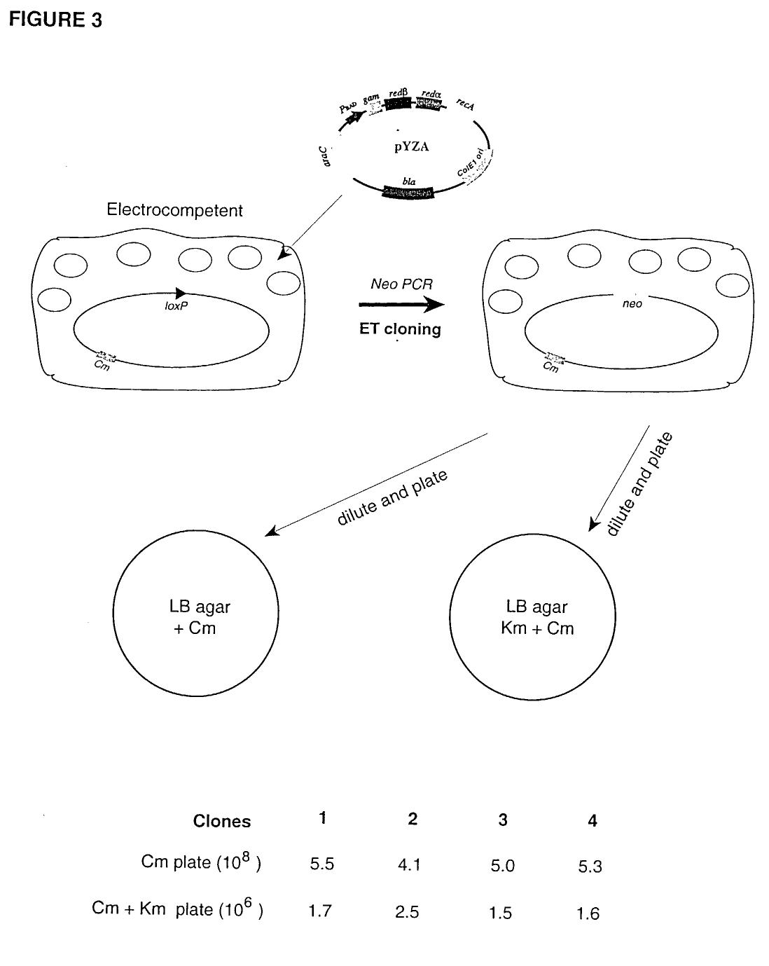

FIG. 3

The vector pYZA was transformed into MLL BAC host cells (HS996, a phage-resistant direct derivative of DH10B, Research Genetics). The cells were made electrocompetent. Then a PCR product of the kanamycin resistance gene flanked by homology arms (60 nt) was introduced into the cell to delete the loxP-site in the BAC backbone. The results of the experiments are shown.

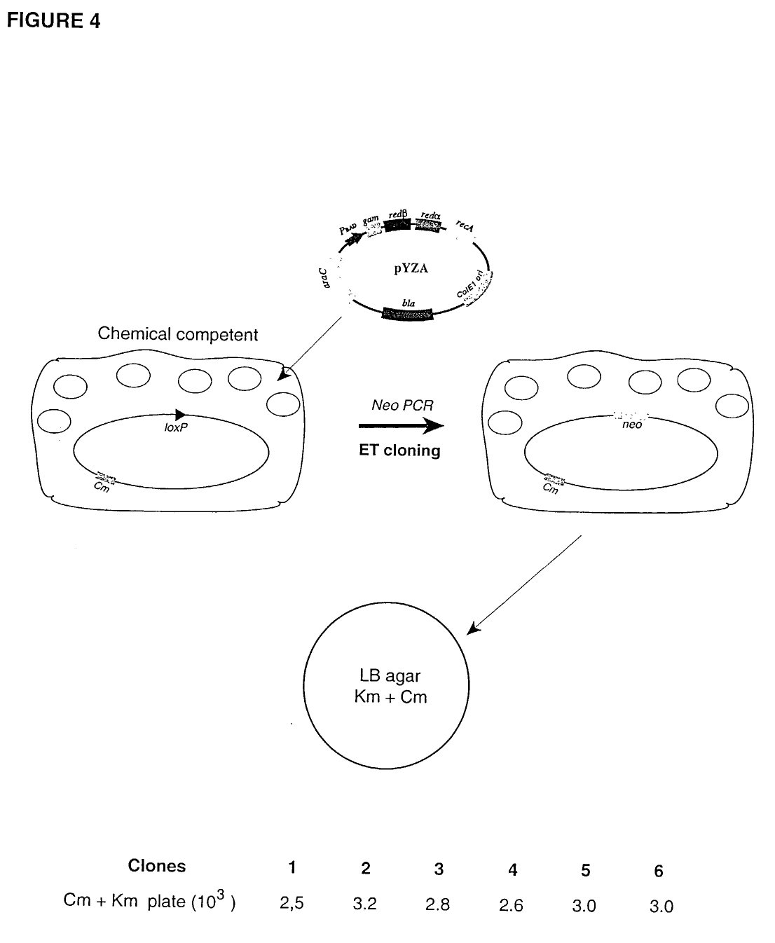

FIG. 4

The host cell and experimental design is as described for FIG. 3. However, the cells were made chemically competent by the rubidium chloride method as follows:

Prepare RbCl chemical competent cells:

a) 0.35 ml of overnight culture were inoculated in 30 ml of LB medium plus amp and cm.

b) induce the recombinase expression with L-arabinose at OD600˜0.15 for 45-60 min.

c) spin down the cells at −5° C., 4,000 rpm for 10 min.

d) resuspend the cells in 15 ml of RbCl buffer on ice and respin.

e) resuspend the cells in 120 μl of RbCl buffer.

f) 40 μl of cells were transferred to an Eppendorf tube and 1.5 μl of a neo PCR product were added.

g) incubate on ice for 10 min.

h) subject the cells to heat-shock at 42° C. for 2.5 min.

i) add 1.0 ml of LB medium and incubate at 37° C. for 75 min. with shaking.

j) plate the cells on Cm plus Km plates.

The results are shown and demonstrate a high number of chloramphenicol/kanamycin resistant clones.

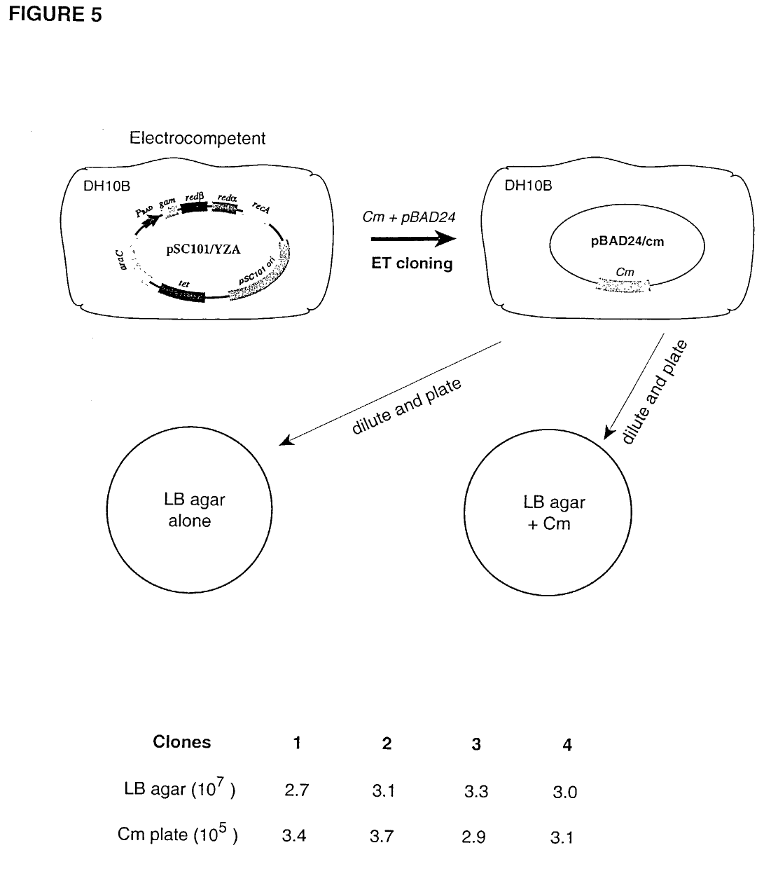

FIG. 5

Electrocompetent E.coli DH10B cells carrying pSC101/YZA were prepared and subjected to cloning as follows:

a) inoculate 0.35 ml of overnight culture in 30 ml of LB medium plus tet (7.5 μg/ml).

b) grow the E.coli cells at 30° C. for 3 hours till OD600˜0.15.

c) add L-arabinose to induce the expression of recombinase for 45-60 min. till OD600˜0.35-0.4.

d) prepare electrocompetent cells as described before.

e) co-transform pBAD24 with a PCR product of the chloramphenicol resistance gene flanked with homology arms (50 nt) to replace the ampicillin resistance gene in pBAD24.

f) plate on Cm plates and incubate at 37° C. overnight, pSC101/YZA will be lost.

The results of the experiment are shown in FIG. 5.

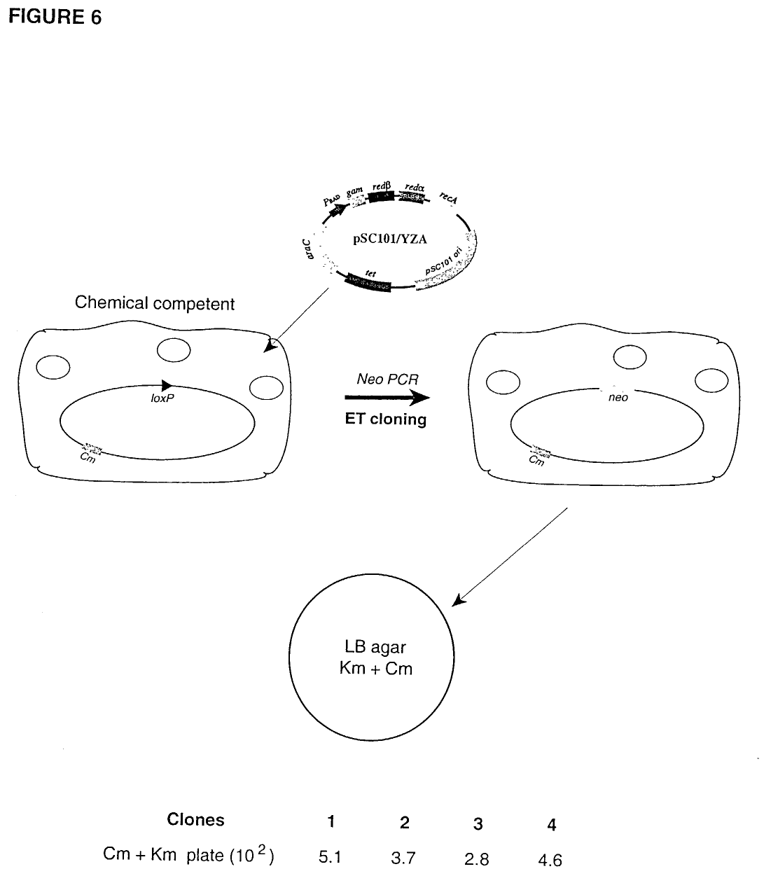

FIG. 6

Chemically competent MLL BAC host cells (HS996) carrying pSC11/YZA were prepared and subjected to cloning as follows:

Prepare the culture:

a) inoculate 0.35 ml of overnight culture in 30 ml of LB medium plus tet (7.5 μg/ml).

b) grow the E.coli cells at 30° C. for 3 hours till OD600˜0.15.

c) add L-arabinose to induce the expression of recombinase for 45-60 min. till OD600˜0.35-0.4.

Prepare RbCl chemically competent cells:

a) spin down the cells at −5° C., 4,000 rpm for 10 min.

b) resuspend the cells in 15 ml of RbCl buffer on ice and respin.

c) resuspend the cells in 120 μl of RbCl buffer.

d) 40 μl of cells were moved in an Eppendorf tube and 1.5 μl of neo PCR (as described in FIG. 3) were added.

e) incubate on ice for 10 min.

f) subject the cells to heat-shock at 42° C. for 2.5 min.

g) add 1.0 ml of LB medium and incubate at 37° C. for 75 min. with shaking.

h) plate on Cm plus Km plates and incubate the plates at 37° C. overnight, (due to its temperature-sensitive origin of replication, pSC101/YZA will be lost).

The results are shown in the figure.

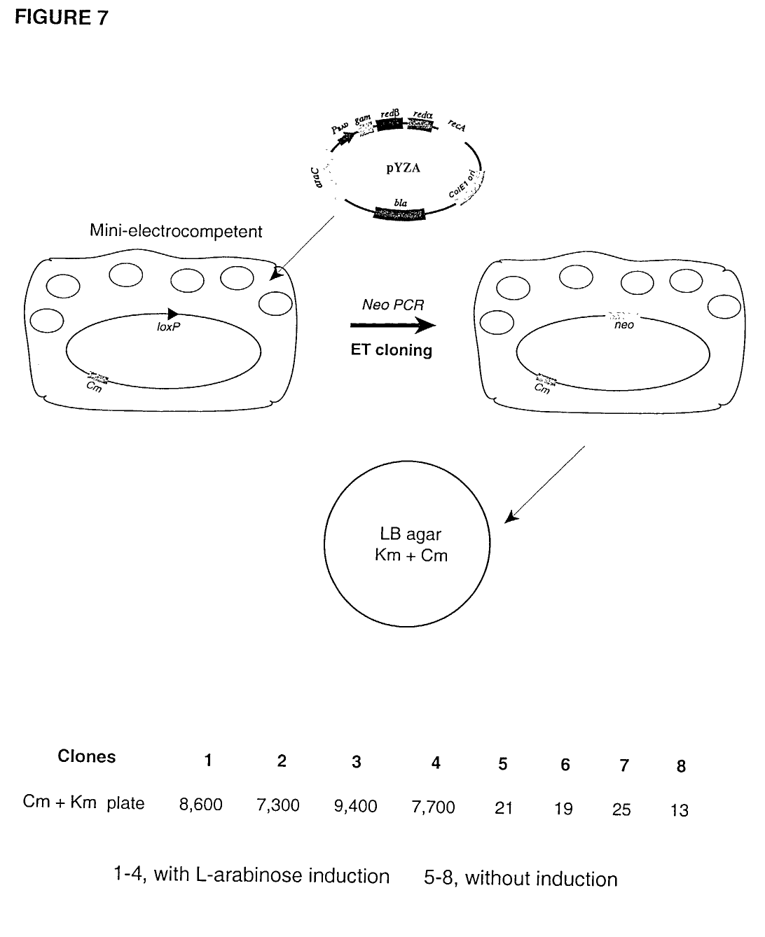

FIG. 7

Transform pYZA into MLL BAC host cells (HS996).

Prepare the competent cells:

a) inoculate a single colony in 1.4 ml LB medium with ampicilin and chloramphenicol in an Eppendorf tube having a hole in the lid.

b) incubate the tube in a heating block at 30° C. with shaking for 3-4 hours till OD600˜0.2.

or

a) add 180 μl of overnight culture in 1.4 ml LB medium with tetracyclin and chloramphenicol in an Eppendorf tube having a hole in the lid.

b) incubate the tube in a heating block at 37° C. with shaking for about 2 hours till OD600˜0.2.

c) add L-arabinose to a final concentration of 0.1% to induce the expression of recombinases.

d) incubate at 37° C. for 45-60 min. till OD600˜0.35-0.4.

e) spin down the cells with the highest speed for 30 sec. in an Eppendorf centrifuge at room temperature.

f) discard the supernatant and put the tube on ice.

g) resuspend the cells in 1.0 ml of 10% ice-cooled glycerol on ice.

h) spin down the cells with the highest speed for 30 sec. and discard the supernatant.

i) resuspend the cells in 1.0 ml of 10% ice-cooled glycerol on ice again.

j) spin down the cells with the highest speed for 30 sec.

k) discard the supernatant by using 1 ml pipette and leave around 20 μl of solution.

l) add 1 μl of neo PCR product (described in FIG. 3) and transfer into an ice-cooled electroporator cuvette (1 mm).

m) electroporate the cells at 1,350 V by using an Eppendorf electroporator.

n) add 1 ml of LB medium and incubate at 37° C. for 75 min.

o) transfer the cells on plates with chloramphenicol and kanamycin.

p) incubate the plates at 37° C. overnight.

The results are shown in the figure.

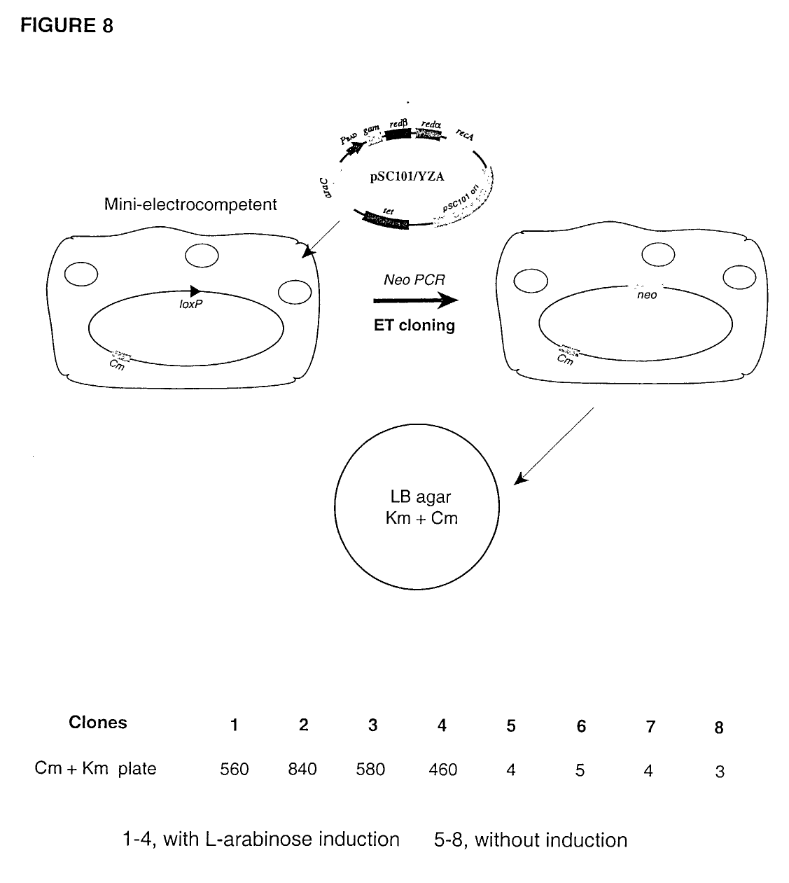

FIG. 8

Transform sPC101/YZA into mouse MLL BAC host cells (HS996). Prepare the competent cells:

a) inoculate a single colony in 1.4 ml LB medium with tetracyclin and chloramphenicol in an Eppendorf tube having a hole in the lid.

b) incubate the tube in a heating block at 30° C. with shaking for about 5 hours till OD600˜0.2.

c) add L-arabinose to a final concentration of 0.1% to induce the expression of recombinases.

d) incubate at 37° C. for 45-60 min. till OD600˜0.35-0.4.

e) spin down the cells with the highest speed for 30 sec. in an Eppendorf centrifuge at room temperature.

f) discard the supernatant and put the tube on ice.

g) resuspend the cells in 1.0 ml of 10% ice-cooled glycerol on ice.

h) spin down the cells with the highest speed for 30 sec. and discard the supernatant.

i) resuspend the cells in 1.0 ml of 10% ice-cooled glycerol on ice again.

j) spin down the cells with the highest speed for 30 sec.

k) discard the supernatant by using 1 ml pipette and leave around 20 μl of solution.

l) add 1 μl of neo PCR product (described in FIG. 3) and transfer into an ice-cooled electroporator cuvette (1 mm).

m) electroporate the cells at 1,350 V by using an Eppendorf electroporator.

n) add 1 ml of LB medium and incubate at 37° C. for 75 min.

o) transfer the cells on plates with chloramphenicol and kanamycin.

p) incubate the plates at 37° C. overnight and pSC101/YZA will be lost.

1. A method for cloning DNA molecules in cells comprising the steps of:
a) providing means for performing homologous recombination via a RecT dependent mechanism within a host cell and/or in vitro,
b) i) contacting in said host cell a first DNA molecule which is capable of being replicated in said host cell with a second DNA molecule under conditions which favour homologous recombination between said first and second DNA molecules and/or
ii) contacting in vitro a first DNA molecule which is capable of being replicated in said host cell with a second DNA molecule under conditions which favour homologous recombination between said first and second DNA molecules and introducing recombined DNA molecules into said host cell, and
c) selecting a host cell in which homologous recombination between said first and second DNA molecules has occurred, wherein a chemically competent host cell and a RecA activity are provided.

2. The method of claim 1 wherein means are provided for performing homologous recombination via a RecET-dependent mechanism within a host cell and/or in vitro.

3. The method of claim 1 or 2, wherein the host cell is capable of expressing a recT gene and optionally a recE gene.

4. The method according to any one of claims 1-3, wherein the recE and recT genes are selected from E.coli recE and recT genes or from λ redα and redβ genes.

5. The method according to any one of claims 1-4, wherein the host cell is transformed with at least one vector capable of expressing recE and/or recT genes.

6. The method of any one of claims 1-5, wherein a RecT protein and/or a RecE protein is contacted outside the host cell with the first and/or the second DNA molecule and then introduced into the host cell.

7. The method of any one of claims 1-6, wherein the host cell is capable of expressing a recA gene.

8. The method of any one of claims 1-7, wherein the host cell is transformed with a vector capable of expressing the recA gene.

9. The method of any one of claims 1-8, wherein a RecA protein is contacted outside the host cell with the first and/or the second DNA molecule and then introduced into the host cell.

10. The method of any one of claims 1-9, wherein the expression of the recE, recT and/or recA genes is under control of a regulatable promoter.

11. The method of claim 4, 5 or 10, wherein the recT gene is overexpressed versus the recE gene.

12. The method of any one of the previous claims wherein the host cell is a chemically competent gram-negative or gram-positive bacterial cell.

13. The method of claim 12, wherein the host cell is an Escherichia coli cell.

14. The method of any one of claims 1-13, wherein the host cell has been made competent by a treatment with calcium chloride, rubidium chloride, glycerol, dimethylsulfoxide, or any combination thereof.

15. The method of any one of claims 1-11, wherein the host cell is a chemically competent eukaryotic cell.

16. The method of claim 15, wherein the host cell has been made competent by treatment with calcium phosphate, DEAE dextran, or any combination thereof.

17. The method according to any one of the previous claims wherein a a RecBC inhibitor activity is provided.

18. The method according to claim 17, wherein the host cell is capable of expressing a recBC inhibitor gene.

19. The method of claim 17 or 18, wherein the recBC inhibitor gene is selected from a redy gene.

20. The method according to any one of claims 17-19, wherein the host cell is transformed with a vector expressing the recBC inhibitor gene.

21. The method according to any one of claims 17-20, wherein the expression of the recBC inhibitor gene is under control of a regulatable promoter.

22. The method according to any one of claims 17 to 21, wherein the host cell is a prokaryotic recBC+cell.

23. The method according to any one of claims 17-22, wherein the RecBC inhibitor is contacted outside the host cell with the first and/or second DNA molecule and then introduced into the host cell.

24. The method according to any one of the previous claims wherein the first DNA molecule is circular.

25. The method according to any one of the previous claims wherein the first DNA molecule is an extrachromosomal DNA molecule containing an origin of replication which is operative in the host cell.

26. The method according to claim 24 or 25, wherein the first DNA molecule is selected from plasmids, cosmids, P1 vectors, BAC vectors and PAC vectors.

27. The method according to any one of claims 1-24, wherein the first DNA molecule is a host cell chromosome.

28. The method according to any one of the previous claims wherein the second DNA molecule is linear.

29. The method according to any one of the previous claims wherein the first and/or second DNA molecules are introduced into the host cells by transformation.

30. The method according to any one of claims 1 to 29, wherein the first and second DNA molecules are introduced into the host cell simultaneously by co-transformation.

31. The method according to any one of claims 1 to 29, wherein the second DNA molecule is introduced into a host cell in which the first DNA molecule is already present.

32. A method according to any one of the previous claims wherein the first DNA molecule contains at least one target site for a site specific recombinase and wherein homologous recombination is detected by removal of said target site.

33. Use of chemically competent cells and the RecT protein, the RecA protein and optionally the RecE protein in a cloning method involving homologous recombination.

34. Use of chemically competent cells capable of expressing the recT gene, the recA gene and optionally the recE gene as a host cell for a cloning method involving homologous recombination.

35. A method for cloning DNA molecules in cells comprising the steps of:
a) providing means for performing homologous recombination via a RecT dependent mechanism within a host cell and/or in vitro,
b) i) contacting in said host cell a first DNA molecule which is capable of being replicated in said host cell with a second DNA molecule under conditions which favour homologous recombination between said first and second DNA molecules and/or
ii) contacting in vitro a first DNA molecule which is capable of being replicated in said host cell with a second DNA molecule under conditions which favour homologous recombination between said first and second DNA molecules and introducing recombined DNA molecules into said host cell, and
c) selecting a host cell in which homologous recombination between said first and second DNA molecules has occurred, wherein a RecA activity is provided.

36. The method of claim 35, wherein means are provided for performing homologous recombination via a RecET-dependent mechanism.

37. The method of claim 35 or 36, wherein the host cell is capable of expressing the recT gene, the recA gene and optionally the recE gene.

38. The method of any one of claims 35-37, wherein the recE, recT and recA genes are under control of a regulatable promoter.

39. The method of any one of claims 35-38, wherein the recE, recT and recA genes are located on at least one extrachromosomal vector.

40. The method of claim 39, wherein the vector has a temperature-sensitive origin.

41. The method of any one of claims 35-40, wherein the RecT protein, the RecA protein and/or the RecE protein are contacted outside the host cell with the first and/or second DNA molecules and then introduced into the host cell.

42. An extrachromosomal vector system capable of expressing recT and recA genes and optionally the recE gene in a host cell.

43. Use of a vector system capable of expressing recT and recA genes and optionally the recE gene in a host cell for a cloning method involving homologous recombination.

44. A reagent kit for cloning comprising
(a) a host cell
(b) means for providing RecT and RecA activity and optionally RecE activity (i) within said host cell, e.g. by expressing recT and recA genes and optionally the recE gene in said host cell and/or (ii) in vitro, e.g. by preincubating at least one of said proteins outside said host cell with DNA molecules, and
(c) a recipient cloning vehicle capable of being replicated in said cell.

45. The reagent kit of claim 44, wherein the host cell is a chemically competent host cell.

46. The reagent kit according to claim 44 or 45, comprising a vector system capable of expressing the recT and recA genes and optionally the recE gene in the host cell.

47. The reagent kit according to any one of claims 44-46 further comprising means for introducing a RecBC inhibitor and/or a site specific recombinase into said host cell, e.g. by expressing a site specific recombinase and/or a recBC inhibitor gene in said host cell, and/or by introducing at least one of said proteins via preincubation with DNA molecules.

48. The reagent kit according to any one of claims 44-47 further comprising adaptor oligonucleotides and/or amplification primers comprising a region of homology to said recipient cloning vehicle.
